(19) United States
(12) Patent Application Publication
(10) Pub. No.: US 2023/0099425 A1
(43) Pub. Date: Mar. 30, 2023

(54) LIFT CLASSIFICATION DEVICE AND SYSTEM

(71) Applicant: StrongArm Technologies, Inc., Brooklyn, NY (US)

(72) Inventors: Michael Patrick Spinelli, Croton, NY (US); SivaSankara Reddy Bommireddy, Secaucus, NJ (US); Jenna Stephenson, Brooklyn, NY (US)

(21) Appl. No.: 17/953,973

(22) Filed: Sep. 27, 2022

Related U.S. Application Data
(60) Provisional application No. 63/249,410, filed on Sep. 28, 2021.

(51) Int. Cl. A61B 5/11; G06K 9/62

(52) U.S. Cl. CPC A61B 5/1118; A61B 5/1116; G06K 9/6223; G06K 9/6276; A61B 2562/0219

(57) ABSTRACT

A system includes a wearable device including an accelerometer configured to record accelerometer data during an activity; a modeling device programmed to determine device acceleration data of the wearable device during the activity based on the accelerometer data, the device acceleration data including x-axis, y-axis, and z-axis acceleration data of the device, translate the device acceleration data to wearer acceleration data of a wearer during the activity, wherein the wearer acceleration data includes at least x-axis and y-axis wearer acceleration data, wherein the y-axis acceleration data of the wearer indicates acceleration along a sagittal axis of the wearer, identify a lift, and utilize a trained lift classification machine learning model to classify the lift as high-risk or low-risk based on a ratio of the x-axis wearer acceleration data to the wearer y-axis acceleration data; and a feedback element configured to provide tangible feedback based on identification of a high-risk lift.

### CROSS-REFERENCE TO RELATED APPLICATION

This application is a Section 111(a) application relating to and claiming the benefit of commonly-owned, co-pending U.S. Provisional Patent Application No. 63/249,410, filed on Sep. 28, 2021, and entitled “LIFT CLASSIFICATION DEVICE AND SYSTEM,” the contents of which are incorporated herein by reference in their entirety.

### FIELD OF THE INVENTION

The present disclosure is related to wearable devices for providing haptic feedback to a wearer based on the wearer's lifting activity. More particularly, the present disclosure is related to wearable devices that distinguish between high-risk lifts and low-risk lifts and provide feedback accordingly.

### BACKGROUND OF THE INVENTION

Sagittal “forward” bending, such as while lifting objects, is a known risk factor that can result in injuries in the workplace. Thus, a technology that is capable of measuring and intervening during bending motions can help reduce the risk of injuries.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1A shows an individual performing a high-risk lift, in which the back muscles are primarily used to lift an object.

FIG. 1B shows an individual performing a low-risk lift, in which the leg muscles are primarily used to lift an object.

FIG. 2 shows a schematic illustration of an exemplary embodiment of a wearable device.

FIG. 3 shows a flowchart of an exemplary embodiment of a method.

FIG. 4 shows exemplary training data used to train an exemplary machine learning classification model that forms a part of the exemplary method of FIG. 3.

### SUMMARY OF THE DISCLOSURE

In some embodiments, a system includes a wearable activity tracking device, a modeling device, and a tangible feedback element; the wearable activity tracking device including an accelerometer, wherein the wearable activity tracking device is configured to be worn by a wearer and to record activity tracking device data during an activity performed by the wearer, and wherein the activity tracking device data includes accelerometer data measured by the accelerometer during the activity; the modeling device including at least one processor, and a non-transient computer memory storing software instructions, wherein, when the at least one processor executes the software instructions, the modeling device is programmed to: receive the activity tracking device data from the wearable activity tracking device, determine activity tracking device acceleration data of the wearable activity tracking device during the activity based on the activity tracking device data, wherein the activity tracking device acceleration data includes at least (a) x-axis acceleration data of the wearable activity tracking device, (b) y-axis acceleration data of the wearable activity tracking device, and (c) z-axis acceleration data of the wearable activity tracking device, translate the activity tracking device acceleration data of the wearable activity tracking device to wearer acceleration data of the wearer during the activity, wherein the wearer acceleration data includes at least: (i) x-axis acceleration data of the wearer, wherein the x-axis acceleration data of the wearer indicates acceleration along a longitudinal axis of the wearer, and (ii) y-axis acceleration data of the wearer, wherein the y-axis acceleration data of the wearer indicates acceleration along a sagittal axis of the wearer; identify a lift performed by the wearer, and utilize a trained lift classification machine learning model to classify the lift as either (i) a high-risk lift or (ii) a low-risk lift, based at least in part on a ratio of the x-axis acceleration data of the wearer at a time of the lift to the y-axis acceleration data of the wearer at the time of the lift; the tangible feedback element configured to provide at least one tangible feedback based on identification of the lift and classification of the lift as the high-risk lift or the low-risk lift, wherein the tangible feedback element is configured to provide a first type of tangible feedback when the lift is identified and is classified as the high-risk lift, and wherein the tangible feedback element is configured not to provide the first type of tangible feedback when the lift is identified and is classified as the low-risk lift.

In some embodiments, when the at least one processor executes the software instructions, the modeling device is further programmed to: determine activity tracking device orientation data of the wearable activity tracking device during the activity based on the activity tracking device data, the activity tracking device orientation data including at least (i) yaw data of the wearable activity tracking device, (ii) pitch data of the wearable activity tracking device, and (iii) roll data of the wearable activity tracking device, and translate the activity tracking device orientation data of the wearable activity tracking device to wearer orientation data of the wearer during the activity, the wearer orientation data including at least pitch data of the wearer, wherein the lift is identified when the pitch data of the wearer at a time of the lift exceeds a threshold pitch. In some embodiments, the threshold pitch is 30 degrees forward from an upright pitch.

In some embodiments, the first type of tangible feedback includes at least one of haptic feedback, visible feedback, or audible feedback.

In some embodiments, the tangible feedback element is configured to provide a second type of tangible feedback when the lift is identified and is classified as the low-risk lift, and wherein the tangible feedback is configured not to provide the second type of tangible feedback when the lift is classified as the high-risk lift. In some embodiments, the second type of tangible feedback includes at least one of haptic feedback, visible feedback, or audible feedback.

In some embodiments, the trained classification machine learning model is based at least in part on one of a K-nearest neighbors algorithm, a support vector machines algorithm, or a convolutional neural network algorithm.

In some embodiments, the tangible feedback element is integrated with the wearable activity tracking device.

In some embodiments, the modeling device is integrated with the wearable activity tracking device.

In some embodiments, the wearable activity tracking device includes an inertial measurement unit.

In some embodiments, a device includes an accelerometer, a modeling device, and a tangible feedback element; the accelerometer being configured to record accelerometer data; the a modeling device including at least one processor, and a non-transient computer memory storing software instructions, wherein, when the at least one processor executes the software instructions, the modeling device is programmed to: receive the accelerometer data from the accelerometer during an activity performed by a wearer of the device, determine device acceleration data of the device during the activity based on the accelerometer data, wherein the acceleration data of the device includes at least (i) x-axis acceleration data of the device, (ii) y-axis acceleration data of the device, and (iii) z-axis acceleration data of the device; translate the device acceleration data of the device to wearer acceleration data of the wearer during the activity, wherein the wearer acceleration data includes at least: (i) x-axis acceleration data of the wearer, wherein the x-axis acceleration data of the wearer indicates acceleration along a longitudinal axis of the wearer, and (ii) y-axis acceleration data of the wearer, wherein the y-axis acceleration data of the wearer indicates acceleration along a sagittal axis of the wearer, identify a lift performed by the wearer, and utilize a trained lift classification machine learning model to classify the lift as either (i) a high-risk lift or (ii) a low-risk lift, based at least in part on a ratio of the x-axis acceleration data of the wearer at a time of the lift to the y-axis acceleration data of the wearer at the time of the lift; and the tangible feedback element being configured to provide at least one tangible feedback based on identification of the lift and classification of the lift as the high-risk lift or the low-risk lift, wherein the tangible feedback element is configured to provide a first type of tangible feedback when the lift is identified and is classified as the high-risk lift, and wherein the tangible feedback element is configured not to provide the first type of tangible feedback when the lift is identified and is classified as the low-risk lift, and wherein the device is configured to be worn by the wearer.

In some embodiments, when the at least one processor executes the software instructions, the modeling device is further programmed to: determine device orientation data of the device during the activity, the device orientation data including at least (i) yaw data of the device, (ii) pitch data of the device, and (iii) roll data of the device, and translate the device orientation data of the device to wearer orientation data of the wearer during the activity, the wearer orientation data including at least pitch data of the wearer, wherein the lift is identified when the pitch data of the wearer at a time of the lift exceeds a threshold pitch. In some embodiments, the threshold pitch is 30 degrees forward from an upright pitch.

In some embodiments, the first type of tangible feedback includes at least one of haptic feedback, visible feedback, or audible feedback.

In some embodiments, the tangible feedback element is configured to provide a second type of tangible feedback when the lift is identified and is classified as the low-risk lift, and wherein the tangible feedback is configured not to provide the second type of tangible feedback when the lift is classified as the high-risk lift. In some embodiments, the second type of tangible feedback includes at least one of haptic feedback, visible feedback, or audible feedback.

In some embodiments, the trained classification machine learning model is based at least in part on one of a K-nearest neighbors algorithm, a support vector machines algorithm, or a convolutional neural network algorithm.

In some embodiments, the device also includes an inertial measurement unit, wherein the inertial measurement unit includes the accelerometer.

In some embodiments, the device is a mobile communication device. In some embodiments, the mobile communication device is a mobile phone.

### DETAILED DESCRIPTION OF THE DRAWINGS

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

The exemplary embodiments relate to wearable devices for monitoring physical activity by the wearer, identifying lifts performed by the wearer, and classifying such lifts as either high-risk or low-risk. Some embodiments of the disclosure may be understood by referring, in part, to the following description and the accompanying drawings, in which like reference numbers refer to the same or like parts.

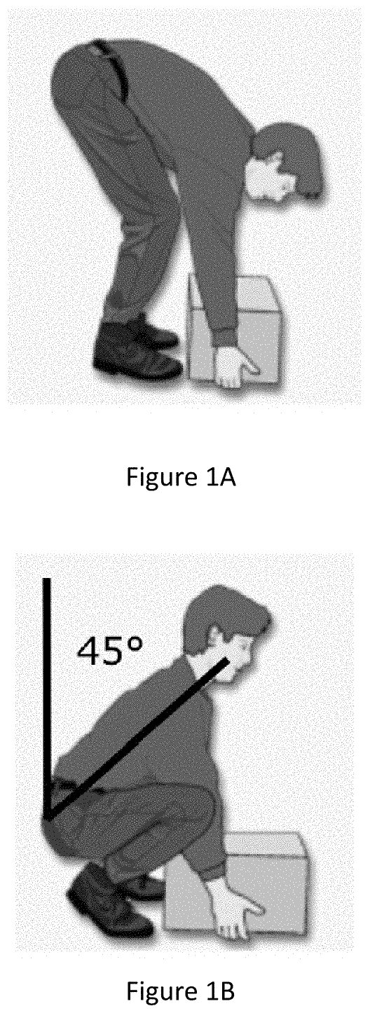

Sagittal “forward” bending, such as while lifting objects, is a known risk factor that can result in injuries in the workplace. Lifting and bending activities can be identified based on an individual's “pitch” (i.e., the angle of the individual's torso with respect to the individual's longitudinal axis) exceeding a threshold value, which may be, for example, 30 degrees. In some cases, a bend detected in this manner can be described as a “bad” lift, during which the individual lifts an object primarily using the individual's back muscles. FIG. 1A illustrates an individual performing a bad lift, in which the individual's torso is bent forward by more than 90 degrees from a vertical orientation. In some cases, a bend detected in this manner can be described as a “good” lift, during which the individual lifts an object primarily using the individual's leg muscles. FIG. 1B illustrates an individual performing a good lift, in which the individual's torso is bent forward by about 45 degrees from a vertical orientation.

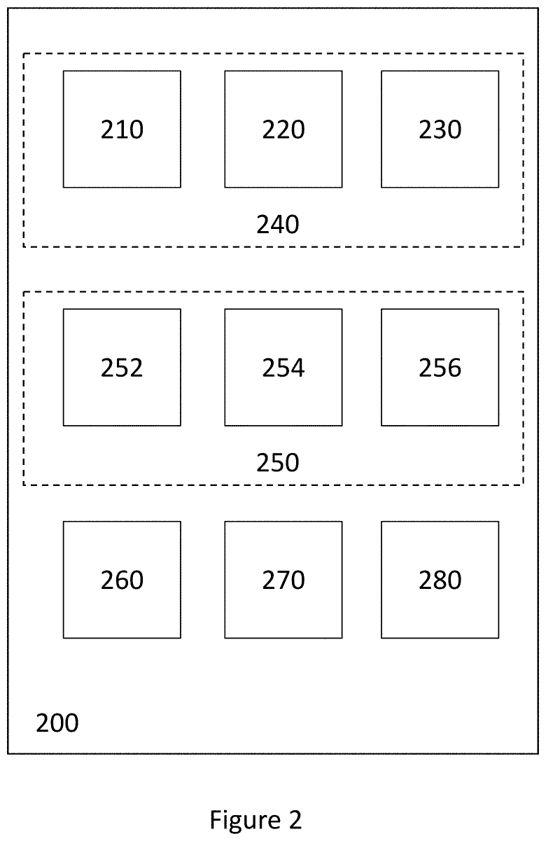

In some embodiments, a wearable device 200 (e.g., a wearable activity tracking device) is operative to provide a wearer (e.g., a person wearing the wearable device 200) with feedback to encourage the wearer to perform good lifts. FIG. 2 schematically illustrates the wearable device 200. In some embodiments, the wearable device 200 is operative to provide the wearer with tangible feedback (e.g., haptic feedback, visible feedback, and/or auditory feedback) when a bad lift is performed. In some embodiments, the wearable device is operative to provide the wearer with a different tangible feedback (e.g., haptic feedback, visible feedback, and/or auditory feedback) when a good lift is performed.

In some embodiments, the wearable device 200 includes at least one sensor. In some embodiments, the wearable device 200 includes an accelerometer 210 (e.g., a triaxial accelerometer). In some embodiments, the wearable device 200 includes a gyroscope 220 (e.g., a triaxial gyroscope). In some embodiments, the wearable device 200 includes a magnetometer 230 (e.g., a triaxial magnetometer). In some embodiments, the wearable device 200 includes two or more of the accelerometer 210, the gyroscope 220, and the magnetometer 230 (e.g., includes the accelerometer 210 and the gyroscope 220, or includes the accelerometer 210 and the magnetometer 230, or includes the gyroscope 220 and the magnetometer 230, or includes the accelerometer 210 and the gyroscope 220 and the magnetometer 230). In some embodiments, the wearable device 200 includes an inertial measurement unit (“IMU”) 240 that includes the accelerometer 210, the gyroscope 220, and the magnetometer 230.

In some embodiments, the wearable device 200 includes an onboard computing system 250. In some embodiments, the computing system 250 includes a microprocessor 252 and a non-transient computer memory 254 storing at least instructions executable by the microprocessor 252 to cause the microprocessor 252 to operate the wearable device 200 as described herein. In some embodiments, the computing system 250 includes one or more communication interfaces 256 (e.g., a wireless communication link such as WiFi hardware and/or a wired communication link such as a USB port) enabling an external computing device to communicate with the computing system 250.

In some embodiments, the microprocessor 252 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the microprocessor 252 may include data-processing capacity provided by the microprocessor. In some embodiments, the microprocessor may include memory, processing, interface resources, controllers, and counters. In some embodiments, the microprocessor may also include one or more programs stored in memory.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

In some embodiments, the non-transient computer memory 254 may include, e.g., a suitable memory or storage solutions for maintaining electronic data representing the activity histories for each account. For example, the non-transient computer memory 254 may include database technology such as, e.g., a centralized or distributed database, cloud storage platform, decentralized system, server or server system, among other storage systems. In some embodiments, the non-transient computer memory 254 may, additionally or alternatively, include one or more data storage devices such as, e.g., a hard drive, solid-state drive, flash drive, or other suitable storage device. In some embodiments, the non-transient computer memory 254 may, additionally or alternatively, include one or more temporary storage devices such as, e.g., a random-access memory, cache, buffer, or other suitable memory device, or any other data storage solution and combinations thereof.

In some embodiments, the non-transient computer memory 254 may include, e.g., instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

In some embodiments, the wearable device 200 includes a haptic feedback element 260. In some embodiments, the haptic feedback element 260 includes a haptic motor. In some embodiments, the wearable device 200 includes a visible feedback element 270. In some embodiments, the visible feedback element 270 includes a display. In some embodiments, the visible feedback element includes one or more indicator lights (e.g., LEDs). In some embodiments, the wearable device 200 includes an audible feedback element 280. In some embodiments, the audible feedback element 280 includes a speaker.

In some embodiments, the wearable device 200 is a device that is specifically designed to monitor the physical activity of the wearer (e.g., the FUSE V5 device commercialized by StrongArm Technologies of Brooklyn, N.Y.). In some embodiments, the wearable device 200 is a general-purpose device such as a mobile phone or other mobile device.

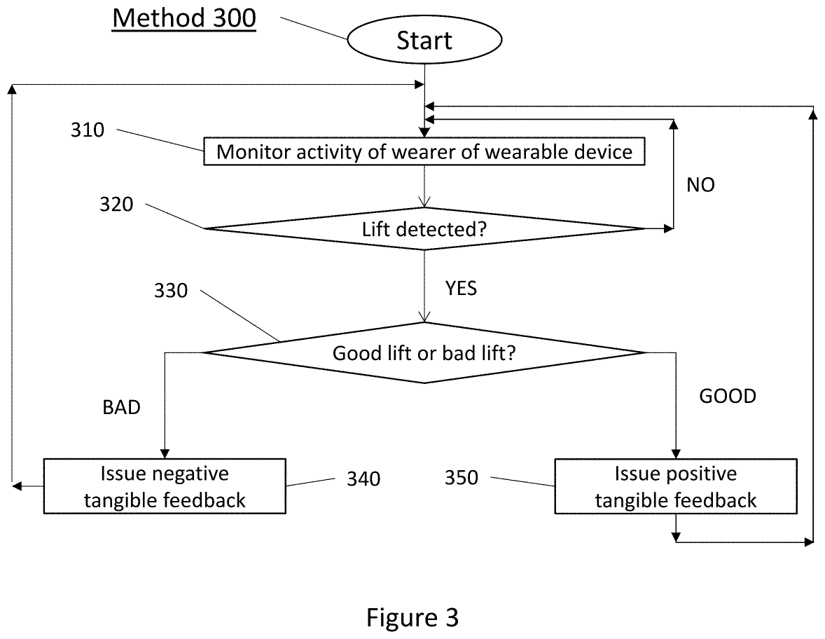

FIG. 3 shows a flowchart of an exemplary method 300 of operation of the wearable device 200. In some embodiments, the method 300 is stored in non-transitory instructions in the memory 254 of the wearable device 200 and is performed by the microprocessor 252 of the wearable device 200 executing such instructions. In step 310, motion of a wearer of the wearable device 200 is continuously monitored while the wearable device 200 is worn. In some embodiments, motion of the wearer is monitored by sensors within the wearable device 200 (e.g., the accelerometer 210, the gyroscope 220, and/or the magnetometer 230). In some embodiments, motion of the wearer is monitored by cameras in an environment in which the wearer is located.

In step 320, it is determined whether the wearer of the wearable device 200 has performed a In some embodiments, monitoring motion of the wearer of the wearable device 200 includes detecting lifts performed by the wearer. In some embodiments, a lift occurs when the wearer's body pitch, which indicates the forward bend of the wearer's torso as compared to the wearer's longitudinal axis, exceeds a threshold pitch. In some embodiments, the threshold pitch is 30 degrees. In some embodiments, the threshold pitch is between 25 and 35 degrees. In some embodiments, the threshold pitch is between 20 degrees and 40 degrees.

In some embodiments, the pitch is monitored through analysis of sensors in the wearable device 200 that include the accelerometer 210 and the gyroscope 220. In some embodiments, orientation of the wearable device 200 is determined based on data recorded by the accelerometer 210 and the gyroscope 220. In some embodiments, the orientation of the wearable device 200 is translated to determine the orientation of the wearer. In some embodiments, the orientation of the wearer includes at least a pitch of the wearer. In some embodiments, the pitch of the wearer is compared to the threshold pitch as described above, and a lift is identified when the pitch of the wearer exceeds the threshold pitch (for example, in embodiments where the threshold pitch is 30 degrees, a lift is identified where the wearer's pitch is more than 30 degrees forward from vertical).

In some embodiments, the orientation of the wearable device 200 at any given moment in time can be described by considering an absolute reference frame of three orthogonal axes X, Y, and Z, defined by the Z-axis being parallel and opposite to the Earth's gravity's downward direction, the X-axis pointing towards the Earth's magnetic north, and the Y-axis pointing in a 90-degree counterclockwise rotation from the Z-axis. In some embodiments, the orientation of the wearable device 200 in space is described as a rotation from the zero-points of this absolute reference frame. In some embodiments, a Tait-Bryan chained rotation (i.e., a subset of Davenport chained rotations) is used to describe the rotation of the wearable device 200 from the zero points of the absolute reference frame to the orientation of the wearable device 200 in space. In some embodiments, the rotation is a geometric transformation which takes the yaw, pitch, and roll angles as inputs and outputs a vector that describes the orientation of the wearable device 200.

In some embodiments, the yaw, pitch, and roll angles that describe the spatial orientation of the wearable device 200 are used to calculate the yaw, pitch, and roll angles that describe the spatial orientation of the body of the individual who is wearing the wearable device 200. In some embodiments, to perform this calculation, it is assumed that the wearable device 200 is rigidly fixed to the initially upright body of the wearer, and the Tait-Bryan chained rotation of the wearable device 200 is applied in reverse order, to the body, instead of to the wearable device 200. In some embodiments, the result of this rotation is a vector which can be considered to be the zero point of the body, to which the yaw, pitch, and roll angles of the wearable device 200 can be applied via a further Tait-Bryan chained rotation to calculate a vector that describes the orientation of the body in space at all times (i.e., a set of YPR values for the body). In some embodiments, a geometric calculation is performed on the set of YPR values for the body to determine the sagittal, twist, and lateral positions. In some embodiments, the sagittal, twist, and lateral positions are determined according to the following equations, with YPR values in degrees: Sagittal=(−1*cos(Roll))*(90-Pitch) Lateral=(−1*sin(Roll))*(90-Pitch).

In some embodiments, the pitch is monitored through analysis of sensors in the wearable device 200 that include only the accelerometer 210. In some embodiments, wherein the y-axis acceleration α_{y} represents acceleration along the wearer's longitudinal axis and the x-axis acceleration αₓ represents acceleration along the wearer's sagittal axis, the pitch angle Θ can be calculated as

$$θ = {tan}^{- 1} ( \frac{{a}_{x}}{{a}_{y}} ) .$$

In some embodiments, the calculated pitch angle is then compared to a threshold pitch to identify a lift (for example, in embodiments where the threshold pitch is 30 degrees, a lift is identified where the wearer's pitch is more than 30 degrees forward from vertical).

In some embodiments, the pitch is monitored through machine vision analysis of images of the wearer of the wearable device 200. In some embodiments, images of the wearer are analyzed to identify the wearer's joints, torso, limbs, etc. In some embodiments, each image of the wearer is analyzed to locate body parts including at least the torso joints and the shoulder joints. In some embodiments, images of the wearer are analyzed using the pose MOVENET detection algorithm forming a part of the TENSORFLOW machine learning library commercialized by Google LLC of Mountain View, Calif. In some embodiments, the key points (e.g., body parts) extracted from a sequence of frames are analyzed by a long short-term memory (“LSTM”) network to determine an activity occurring during the sequence of frames. More particularly, in some embodiments, the key points are analyzed by an LSTM network to determine if a bend occurred during the sequence of frames. In some embodiments, if a bend occurred, the results of such analysis are further analyzed to calculate a pitch angle of the wearer's torso. In some embodiments, the displacement of the torso joints and the shoulder joints from an initial position to the new position in three dimensions is calculated to provide the displacement of the torso, e.g., the pitch angle. In some embodiments, the calculated pitch angle is then compared to a threshold pitch to identify a lift (for example, in embodiments where the threshold pitch is 30 degrees, a lift is identified where the wearer's pitch is more than 30 degrees forward from vertical).

In some embodiments, once a lift has been detected in step 320, in step 330 the lift is classified as either a good lift (e.g., a low-risk lift) or a bad lift (e.g., a high-risk lift). In some embodiments, a lift is classified as either a good lift or a bad lift by utilizing a trained machine learning classification model to classify the lift based on the ratio of the wearer's x-axis acceleration αₓ (e.g., acceleration along the longitudinal axis of the wearer, such as “downward” acceleration) to the wearer's y-axis acceleration α_{y} (e.g., acceleration along the sagittal axis of the wearer, such as “forward” acceleration). In some embodiments, the ratio of the wearer's x-axis acceleration to y-axis acceleration is indicative the presence or absence of bending at the knees during a lift, because the wearer accelerates downward (i.e., in the x direction) when bending at the knees). Therefore, this ratio is indicative of whether a lift is a low-risk or high-risk lift. In some embodiments, αₓ and α_{y} are determined by measuring the x-axis acceleration, y-axis acceleration, and z-axis acceleration of the wearable device 200, and translating from the wearable device 200 frame of reference to the wearer frame of reference to determine αₓ and α_{y}. In some embodiments, translation of acceleration from the wearable device 200 frame of reference to the wearer frame of reference is accomplished using at least one Tait-Bryan rotation in the manner described above with reference to translation of orientation.

In some embodiments, the machine learning classification module is based at least in part on one of a K-nearest neighbors algorithm, a support vector machines algorithm, or a convolutional neural network algorithm.

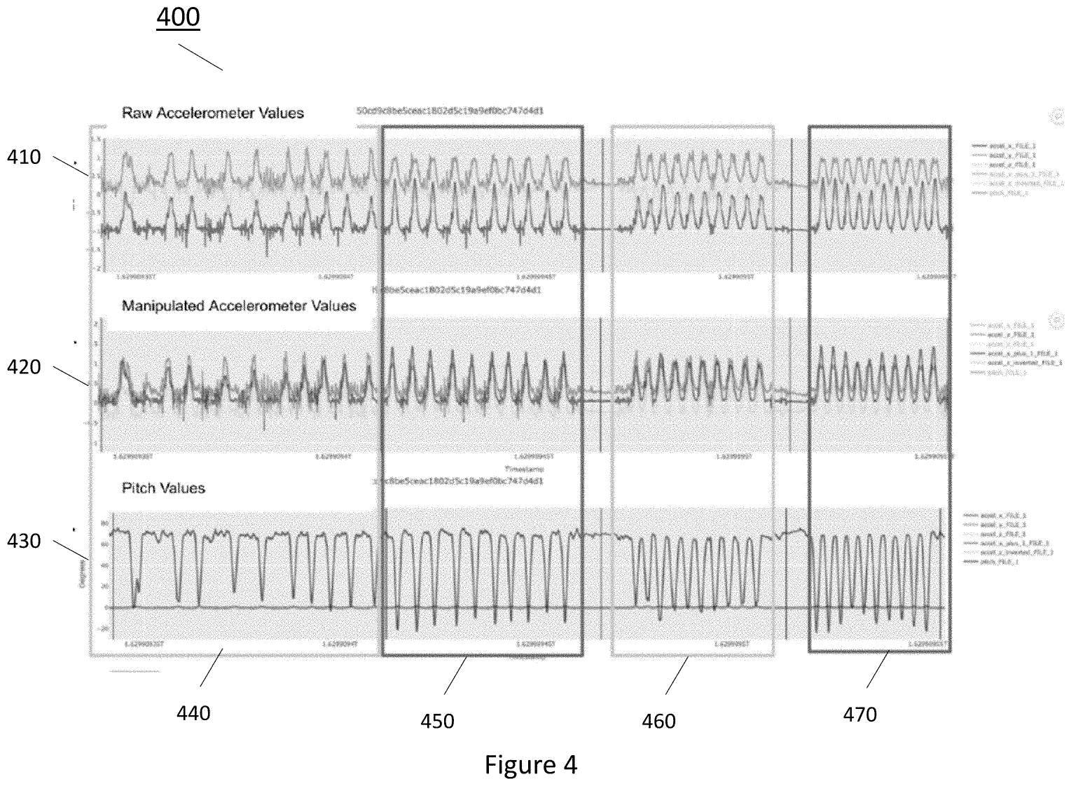

In some embodiments, the machine learning classification module is trained using training data relating to a sequence of lifts performed by one or more individuals. In some embodiments, during such a sequence of lifts, α_{y} and αₓ are determined for the individual (for example, in the manner described above) and each lift is manually identified as either a good lift or a bad lift as part of the training. FIG. 4 shows sample training data 400 for the machine learning classification module. The training data 400 includes a time series 410 of raw accelerometer values (e.g., accelerometer values for a wearable device), a time series 420 of manipulated accelerometer values (e.g., accelerometer values for an individual), and a time series 430 of pitch values for the individual. The training data 400 shows, for each of the three time series 410, 420, and 430, a first set 440 of good lifts, a second set 450 of bad lifts, a third set 460 of good lifts, and a fourth set 470 of bad lifts.

In some embodiments, if it is determined in step 330 that the lift was a bad lift, then the method proceeds to step 340. In step 340, negative tangible feedback is provided by the wearable device 200. In some embodiments, the negative tangible feedback includes negative haptic feedback provided by the haptic feedback element 260. In some embodiments, the negative tangible feedback includes negative visible feedback provided by the visible feedback element 270. In some embodiments, the negative visible feedback is color-coded so as to indicate negative feedback (e.g., includes a red light). In some embodiments, the negative tangible feedback includes negative audible feedback provided by the audible feedback element 280.

In some embodiments, if it is determined in step 330 that the lift was a good lift, then the method proceeds to step 350. In step 350, positive tangible feedback is provided by the wearable device 200. In some embodiments, the positive tangible feedback includes positive haptic feedback provided by the haptic feedback element 260. In some embodiments, the positive tangible feedback includes positive visible feedback provided by the visible feedback element 270. In some embodiments, the positive visible feedback is color-coded so as to indicate positive feedback (e.g., includes a green light). In some embodiments, the positive tangible feedback includes positive audible feedback provided by the audible feedback element 280.

In some embodiments, the negative feedback provided in step 340 is negative haptic feedback and the positive feedback provided in step 350 is positive audible feedback. In some embodiments, the negative feedback provided in step 340 is negative haptic feedback and the positive feedback provided in step 350 is positive visible feedback. In some embodiments, the negative feedback provided in step 340 includes negative haptic feedback and negative audible feedback and the positive feedback provided in step 350 is positive audible feedback. In some embodiments, the negative feedback provided in step 340 includes negative haptic feedback and negative visible feedback and the positive feedback provided in step 350 is positive visible feedback.

In some embodiments, following either negative feedback in step 340 or positive feedback in step 350, the method 300 returns to step 310, and monitoring of the wearer of the wearable device 200 continues. In some embodiments, monitoring continues for as long as the wearer continues to wear the wearable device 200. In some embodiments, monitoring continues until the wearer disengages the wearable device 200 (e.g., removes the wearable device 200, powers off the wearable device 200, or instructs the wearable device 200 to cease monitoring).

In some embodiments, by providing feedback on lifting behavior, the wearable device 200 decreases the risk of injury and improves workplace safety. In some embodiments, by distinguishing good lifts from bad lifts, the wearable device 200 provides improved accuracy of lift alerting. In some embodiments, by distinguishing good lifts from bad lifts, the wearable device provides a technical improvement to solutions that identify lifting or bending behavior based on pitch but do not take the presence or absence of bending at the knees into account.

While a number of embodiments of the present invention have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

What is claimed is:

1. A system, comprising:
a wearable activity tracking device including an accelerometer,
wherein the wearable activity tracking device is configured to be worn by a wearer and to record activity tracking device data during an activity performed by the wearer, and
wherein the activity tracking device data comprises accelerometer data measured by the accelerometer during the activity;
a modeling device comprising:
at least one processor, and
a non-transient computer memory, storing software instructions,
wherein, when the at least one processor executes the software instructions, the modeling device is programmed to:
a) receive the activity tracking device data from the wearable activity tracking device,
b) determine activity tracking device acceleration data of the wearable activity tracking device during the activity based on the activity tracking device data, wherein the activity tracking device acceleration data comprises at least (a) x-axis acceleration data of the wearable activity tracking device, (b) y-axis acceleration data of the wearable activity tracking device, and (c) z-axis acceleration data of the wearable activity tracking device,
c) translate the activity tracking device acceleration data of the wearable activity tracking device to wearer acceleration data of the wearer during the activity, wherein the wearer acceleration data comprises at least:
(i) x-axis acceleration data of the wearer, wherein the x-axis acceleration data of the wearer indicates acceleration along a longitudinal axis of the wearer, and
(ii) y-axis acceleration data of the wearer, wherein the y-axis acceleration data of the wearer indicates acceleration along a sagittal axis of the wearer,
d) identify a lift performed by the wearer, and
e) utilize a trained lift classification machine learning model to classify the lift as either (i) a high-risk lift or (ii) a low-risk lift, based at least in part on a ratio of the x-axis acceleration data of the wearer at a time of the lift to the y-axis acceleration data of the wearer at the time of the lift; and
a tangible feedback element configured to provide at least one tangible feedback based on identification of the lift and classification of the lift as the high-risk lift or the low-risk lift,
wherein the tangible feedback element is configured to provide a first type of tangible feedback when the lift is identified and is classified as the high-risk lift, and wherein the tangible feedback element is configured not to provide the first type of tangible feedback when the lift is identified and is classified as the low-risk lift.

2. The system of claim 1, wherein, when the at least one processor executes the software instructions, the modeling device is further programmed to:
determine activity tracking device orientation data of the wearable activity tracking device during the activity based on the activity tracking device data, the activity tracking device orientation data including at least (i) yaw data of the wearable activity tracking device, (ii) pitch data of the wearable activity tracking device, and (iii) roll data of the wearable activity tracking device, and
translate the activity tracking device orientation data of the wearable activity tracking device to wearer orientation data of the wearer during the activity, the wearer orientation data comprising at least pitch data of the wearer,
wherein the lift is identified when the pitch data of the wearer at a time of the lift exceeds a threshold pitch.

3. The system of claim 2, wherein the threshold pitch is 30 degrees forward from an upright pitch.

4. The system of claim 1, wherein the first type of tangible feedback comprises at least one of haptic feedback, visible feedback, or audible feedback.

5. The system of claim 1, wherein the tangible feedback element is configured to provide a second type of tangible feedback when the lift is identified and is classified as the low-risk lift, and wherein the tangible feedback is configured not to provide the second type of tangible feedback when the lift is classified as the high-risk lift.

6. The system of claim 5, wherein the second type of tangible feedback comprises at least one of haptic feedback, visible feedback, or audible feedback.

7. The system of claim 1, wherein the trained classification machine learning model is based at least in part on one of a K-nearest neighbors algorithm, a support vector machines algorithm, or a convolutional neural network algorithm.

8. The system of claim 1, wherein the tangible feedback element is integrated with the wearable activity tracking device.

9. The system of claim 1, wherein the modeling device is integrated with the wearable activity tracking device.

10. The system of claim 1, wherein the wearable activity tracking device includes an inertial measurement unit.

11. A device, comprising:
an accelerometer configured to record accelerometer data;
a modeling device comprising:
at least one processor, and
a non-transient computer memory storing software instructions,
wherein, when the at least one processor executes the software instructions, the modeling device is programmed to:
a) receive the accelerometer data from the accelerometer during an activity performed by a wearer of the device,
b) determine device acceleration data of the device during the activity based on the accelerometer data, wherein the acceleration data of the device comprises at least (i) x-axis acceleration data of the device, (ii) y-axis acceleration data of the device, and (iii) z-axis acceleration data of the device,
c) translate the device acceleration data of the device to wearer acceleration data of the wearer during the activity, wherein the wearer acceleration data comprises at least:
(i) x-axis acceleration data of the wearer, wherein the x-axis acceleration data of the wearer indicates acceleration along a longitudinal axis of the wearer, and
(ii) y-axis acceleration data of the wearer, wherein the y-axis acceleration data of the wearer indicates acceleration along a sagittal axis of the wearer,
d) identify a lift performed by the wearer, and
e) utilize a trained lift classification machine learning model to classify the lift as either (i) a high-risk lift or (ii) a low-risk lift, based at least in part on a ratio of the x-axis acceleration data of the wearer at a time of the lift to the y-axis acceleration data of the wearer at the time of the lift; and
a tangible feedback element configured to provide at least one tangible feedback based on identification of the lift and classification of the lift as the high-risk lift or the low-risk lift,
wherein the tangible feedback element is configured to provide a first type of tangible feedback when the lift is identified and is classified as the high-risk lift, and wherein the tangible feedback element is configured not to provide the first type of tangible feedback when the lift is identified and is classified as the low-risk lift,
wherein the device is configured to be worn by the wearer.

12. The device of claim 11, wherein, when the at least one processor executes the software instructions, the modeling device is further programmed to:
determine device orientation data of the device during the activity, the device orientation data including at least (i) yaw data of the device, (ii) pitch data of the device, and (iii) roll data of the device, and
translate the device orientation data of the device to wearer orientation data of the wearer during the activity, the wearer orientation data comprising at least pitch data of the wearer,
wherein the lift is identified when the pitch data of the wearer at a time of the lift exceeds a threshold pitch.

13. The device of claim 12, wherein the threshold pitch is 30 degrees forward from an upright pitch.

14. The device of claim 11, wherein the first type of tangible feedback comprises at least one of haptic feedback, visible feedback, or audible feedback.

15. The device of claim 11, wherein the tangible feedback element is configured to provide a second type of tangible feedback when the lift is identified and is classified as the low-risk lift, and wherein the tangible feedback is configured not to provide the second type of tangible feedback when the lift is classified as the high-risk lift.

16. The device of claim 15, wherein the second type of tangible feedback comprises at least one of haptic feedback, visible feedback, or audible feedback.

17. The device of claim 11, wherein the trained classification machine learning model is based at least in part on one of a K-nearest neighbors algorithm, a support vector machines algorithm, or a convolutional neural network algorithm.

18. The device of claim 11, further comprising an inertial measurement unit, wherein the inertial measurement unit includes the accelerometer.

19. The device of claim 11, wherein the device is a mobile communication device.

20. The device of claim 19, wherein the mobile communication device is a mobile phone.
